(19) United States
(12) Patent Application Publication
(10) Pub. No.: US 2017/0209652 A1
(43) Pub. Date: Jul. 27, 2017

(54) Needle Guard

(71) Applicant: NOBLE HOUSE GROUP PTY. LTD., Chelsea Heights, Victoria (AU)

(72) Inventor: Chris Whelan, Leichhardt (AU)

(21) Appl. No.: 15/328,502

(22) PCT Filed: Jul. 24, 2015
(86) PCT No.: PCT/AU2015/000439

§ 371 Date: Jan. 24, 2017

Related U.S. Application Data
(60) Provisional application No. 62/028,403, filed on Jul. 24, 2014.

(51) Int. Cl. A61M 5/32; A61M 5/158

(52) U.S. Cl. CPC A61M 5/3221; A61M 5/3243; A61M 5/158; A61M 2005/3224; A61M 2005/3247

(57) ABSTRACT

A needle guard (10) for use with a needle assembly (12) having a needle (16) extending from a forward end of a needle block (18), the guard (10) comprises a hollow sleeve portion (26) having a front end (20) with an aperture (24) therein through which the needle assembly (12) may be retracted to a retracted position, in which the free end of the needle lies within the hollow sleeve portion. The guard (10) has a retaining portion into which at least a part of the needle assembly may be withdrawn to a retracted position, at least one locking mechanism (36) to retain the needle assembly in the retaining portion and at least one deflector (36, 102) that engages the needle (16) and/or the needle block (18) to deflect the needle (16) away from the aperture (24) when in the retracted position.

### FIELD OF INVENTION

This invention relates to needle guards and more particularly to guards into which a needle assembly having a needle at an end is withdrawn.

### BACKGROUND

Needle guards are devices into which a needle of a needle assembly can be withdrawn after use. Once fully withdrawn the needle is locked within the guard and cannot be accidentally extended, thus preventing accidental needle stick injury once it is within the guard.

### SUMMARY OF THE INVENTION

The present invention provides a needle guard for use with a needle assembly having a needle extending from a needle block having a forward end, the guard comprising:
a hollow sleeve portion having a front end with an aperture therein into which the needle may be retracted to a retracted position, in which the free end of the needle lies within the hollow sleeve portion;
a retaining portion into which at least a part of the needle assembly may be withdrawn to a retracted position;
at least one locking mechanism to retain the needle in the retaining portion;
at least one deflector that cooperates with the needle assembly to deflect the needle away from the aperture when in the retracted position.

Preferably the at least one deflector engages the needle and/or the needle block.

The deflector may comprise at least one deflector surface against which at least a portion of the needle and/or the needle block bears when in the retracted position.

In one form the at least one deflector surface may comprise a part of at least one flexible arm that engages a portion of the needle and/or the needle block and, in the retracted position, applies a deflecting force to the engaged portion.

The deflector surface may be curved.

The at least one flexible arm may comprise the at least one locking mechanism or the at least one locking mechanism may comprise the at least one flexible arm.

The at least one flexible arm may be separate from the at least one locking mechanism.

The at least one deflector surface may include a surface portion of the retaining portion. The surface portion may be complementary to the surface of the needle block so that when the needle block bears against the surface portion the free end of the needle is located away from the aperture.

The needle guard may include a retaining member to provide retention of the needle block in the retaining portion.

The hollow sleeve portion preferably has an aperture through which the needle and needle block pass when moving to the retracted position.

The aperture may be at least partially defined by at least one wall. In the retracted position the end of the needle preferably lies behind the at least one wall.

The at least one wall may be relatively rigid and/or immovable as the needle and needle block pass through the aperture.

The aperture may be at least partially defined by at least one arm. The at least one arm may be relatively flexible and/or movable as the needle and needle block pass through the aperture. Use of flexible and/or movable arm(s) allows the aperture to allow passage of a needle block larger than the normal size of aperture

The at least one wall preferably extends rearwards and inwards.

The at least one arm preferably extends rearwards and inwards.

### BRIEF DESCRIPTION OF THE DRAWINGS

FIG. 1 is a perspective view from above of a needle guard according to an embodiment of the invention;

FIG. 2 is a perspective view from below of the needle guard of FIG. 1;

FIG. 3 is an end view of the needle guard of FIG. 1;

FIG. 4 is a plan view of the needle guard of FIG. 1;

FIG. 5 is a side view of the needle guard of FIG. 1;

FIG. 6 is a perspective sectional view taken along line AA of FIG. 3 looking forward;

FIG. 7 is a perspective sectional view taken along line AA of FIG. 3 looking rearward;

FIG. 8 is a side sectional view taken along line AA of FIG. 3;

FIG. 9 is a plan view of the needle guard of FIG. 1 mounted on a needle assembly with the needle assembly in a non-retracted position;

FIG. 10 is a side view of the needle guard of FIG. 1 mounted on a needle assembly with the needle assembly in a non-retracted position;

FIG. 11 is a longitudinal cross sectional view of the needle guard and the needle assembly in a non-retracted position;

FIG. 12 is a plan view of the needle guard of FIG. 1 mounted on a needle assembly with the needle assembly in a retracted position;

FIG. 13 is a side view of the needle guard of FIG. 1 mounted on a needle assembly with the needle assembly in a retracted position;

FIG. 14 is a longitudinal cross sectional view of the needle guard and the needle assembly in a retracted position.

FIG. 15 is a longitudinal cross sectional view a needle guard according to anther embodiment of the invention with a needle assembly in a retracted position.

FIG. 16 is a side view of a needle guard with an alternate aperture arrangement according to another embodiment of the invention.

FIG. 17 is an end view of a needle guard with an alternate aperture arrangement.

FIG. 18 is a cross sectional view of the needle guard of FIG. 16 taken along line AA of FIG. 17.

FIG. 19 is a cross sectional view of the needle guard of FIG. 16 taken along line BB of FIG. 17.

FIG. 20 is a detail cross sectional view of the portion of needle guard of FIG. 16 circled C in FIG. 17.

### DETAILED DESCRIPTION OF PREFERRED AND OTHER EMBODIMENTS

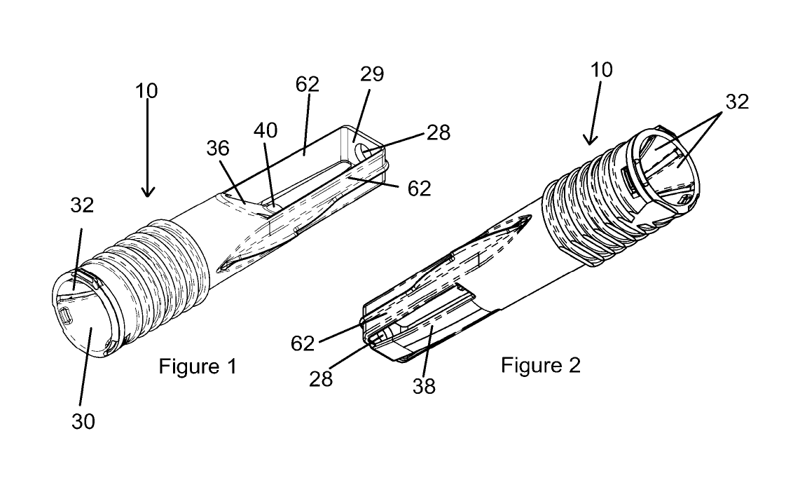

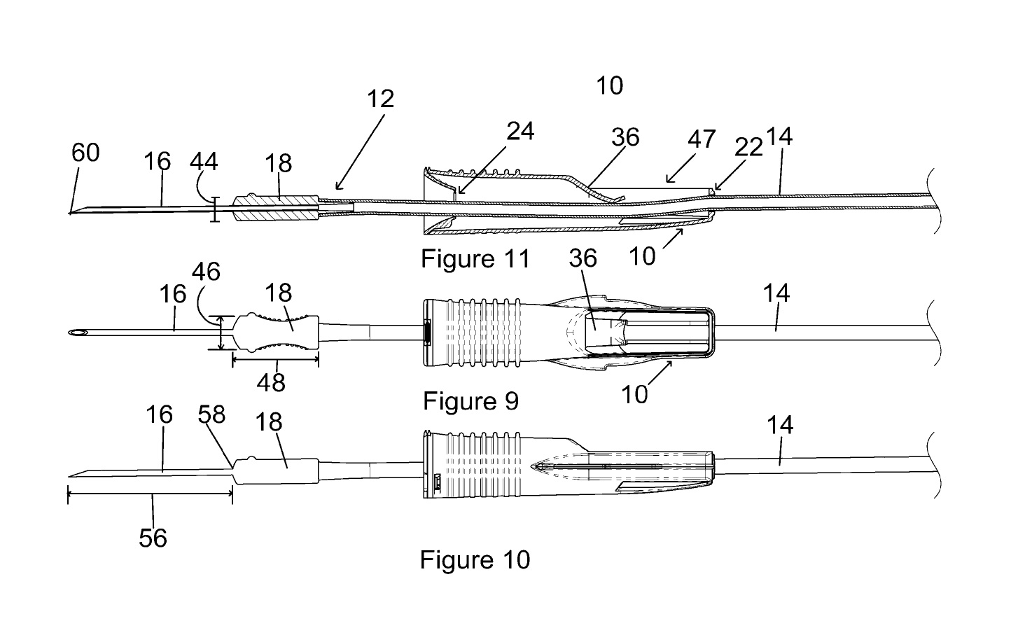

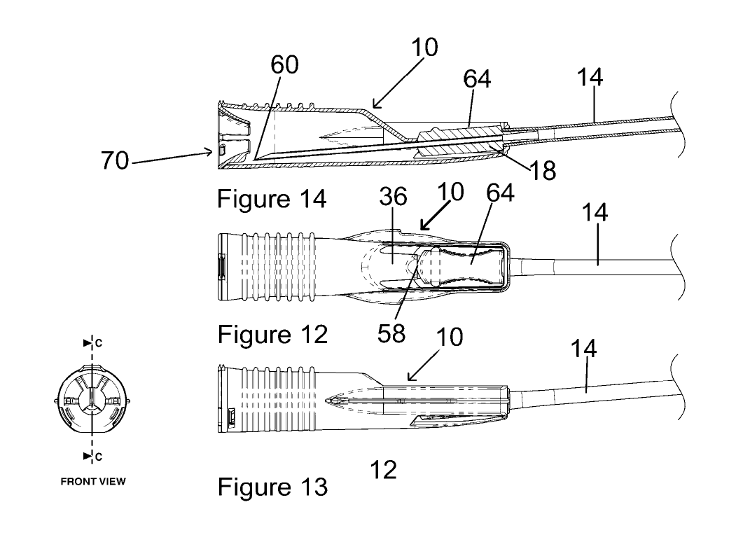

Referring to FIGS. 1 to 14 there is shown a needle guard 10 according to a first embodiment of the invention. The needle guard is adapted to be mounted on a needle assembly 12, as shown in FIGS. 9-14. The needle assembly 12 comprises a conduit 14 and a needle 16 mounted on a needle block 18 with the conduit connected to the needle block 18.

The needle guard 10 has a front end 20 and a rear end 22. The front end 20 has a front aperture 24 that communicates with hollow portion 26. The rear end has rear aperture 28 in rear wall 29.

As seen in FIGS. 9 to 13, the needle guard 10 may be mounted on the needle assembly 12, with the conduit 14 passing through front and rear apertures 24, 28 and the needle 16 and needle block 18 located forward of the front end 20.

The rear aperture 28 is sized to be smaller than the needle block so the needle block cannot pass through the aperture 28. The rear aperture 28 may be an interference fit or a loose fit on the conduit 14. An interference fit allows the needle guard 10 to be slid along the conduit 14 and retained that position by friction on the conduit 14.

The needle guard has a flexible arm 36 that ends rearwards from the rear of the hollow portion 26 toward the base 38 of the guard 10. The arm 36 preferably has an end portion 40. In the un-deflected state the end portion 40 may be generally parallel to the bottom 38 of the needle guard. The arm 36 acts as a deflector and/or a locking mechanism for the needle assembly 12 when it is withdrawn into the guard 10.

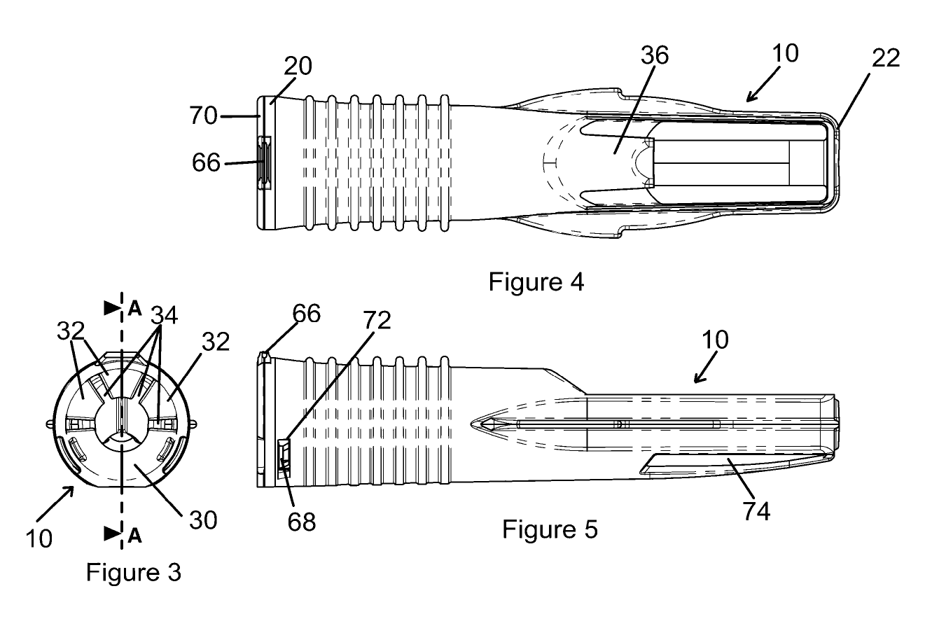

The front aperture 24 is generally circular (see FIG. 3) and is defined by wall 30 and arms 32 that extend inwards and rearwards from the front end 20. The ends of the wall 30 and arms 32 define the generally circular aperture 24. The aperture 24 does not need to be circular.

As best seen in FIG. 3, the wall 30 extends over about 180 degrees and defines about one half of the aperture 24. In the embodiment shown there are three arms 32 defined by slots 34 between themselves and between wall 30. Because the three arms 32 only extend about 60 degrees around the aperture 24 they are relatively flexible. In contrast, because the wall 30 extends over about 180 degrees it is relatively inflexible.

Instead of wall 30 partially defining the aperture 24, a plurality of arms 32 may define the aperture 24. In such an embodiment the arms 32 extend 360 degrees around the aperture 24. There may be more or less than six arms. The arms 32 may be equally sized and/or equally spaced around aperture but do not need to be. The arms 30 do not need to have equal length. The arms 30 do not need to have flexibility.

The needle guard is intended to receive the needle 16 within the hollow portion 26 after use.

After use the needle guard 10 is slid along the conduit 14 so that needle block 18 and needle 16 pass through the aperture 24.

The opening 24 is preferably sized so that than the needle block 18 causes one or more of the arms to deflect as it passes through the opening 24. This prevents accidental retraction of the needle block 18 into the hollow portion 26. However, this is not critical and the opening 24 may be sized so that the needle block does not cause deflection of the arms 32. Where deflection of arms 32 is necessary to draw the needle block 18 into the needle guard the arms 32 will resist or prevent forward removal once the needle block is fully within the hollow portion 26. Use of a circular aperture allows use with a needle block that is circular or non-circular in end view. It also allows the needle block to enter at any rotation.

Continued movement of the needle guard toward the needle causes the needle block 18 to move rearward and engage the flexible arm 36. The distance between bottom 38 and the nearest point 42 of the arm is less than the smallest dimension of the needle block. Where the needle block is generally circular, this would be the diameter of the needle block. Where the needle block is generally rectangular, this would be the smaller of either the height 44 or width 46.

A non-circular needle block contacting the arm 36 will contact the arm 36 off axis if rotated slightly and the arm will tend to cause rotation of the guard 10 relative to the block 18 to align the block with the guard.

The arm is deflected upwards by the needle block, allowing the needle block 18 to enter retaining portion 47

The needle block can thus be drawn rearwards fully past the arm 36 until its rearward motion is limited by rear wall 29 contacting the end of the needle block or the conduit 14 engaging in aperture 28.

The needle block has a length 48 and the distance 50 between the rearmost end 52 of arm 36 and the rear wall 29 is greater than the length 48 and sufficient to allow the needle block to be fully retracted into retaining portion 47.

Once the front end 58 of the needle block has passed the end 52 of the arm 36, the arm moves towards the base to lie in front of the needle block 18, as seen in FIG. 14. In this position the arm 36 prevents forward motion of the needle assembly.

The distance 54 between the rearmost end 52 of arm 36 and aperture 24 (defined by the rearmost portions of wall 30 and arms 32) is greater than the distance 56 between the front end 58 of the needle block and the end 60 of the needle 16.

Accordingly, once the front end 58 of the needle block has passed the end of the arm 36, the free end 60 of the needle will be within the hollow portion 26 and not overlapping the wall 32 or arms 32.

Preferably, the arm 36 is configured so that it bears against the needle and at that position is still deflected from its “rest” position. The arm 36 thus applies a downwards force to the needle and tilts or deflects the needle block and needle so the free end 60 of the needle is off axis, as seen in FIG. 14, and not aligned with the aperture 24. Thus, insertion of an object (such as a person's finger) through the aperture is unlikely to result in contact with the free end 60.

The wall 30, being relatively rigid, also serves as a barrier to prevent access to the needle 16. Insertion of a finger into the aperture 24 will result in deflection of arms 32 and the finger will remain above wall 30.

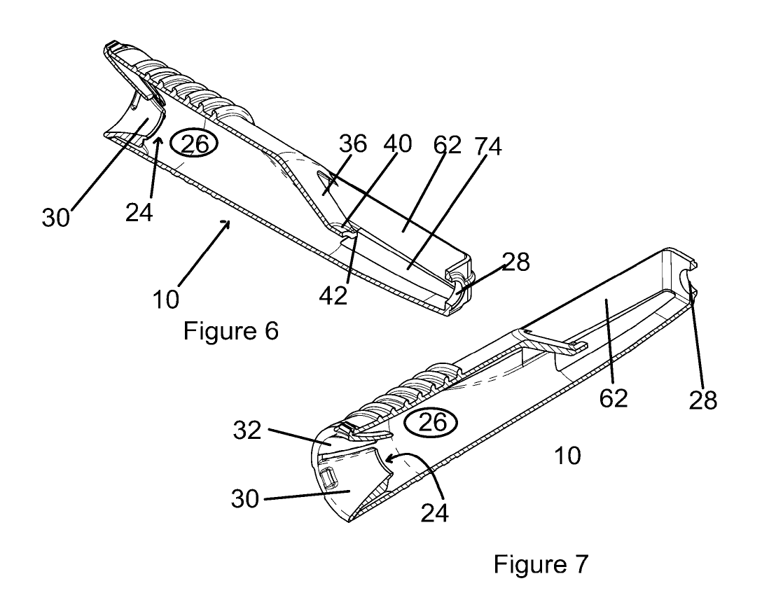

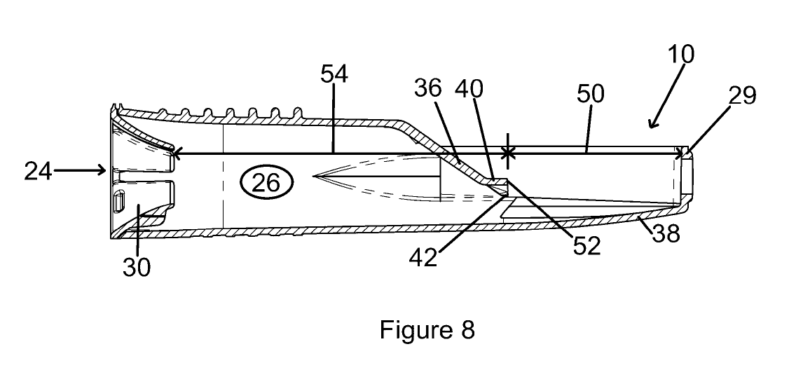

The end portion 40 of the arm 36 is preferably curved when viewed along the axis (see FIGS. 6 and 8) and accordingly when bearing against the needle will tend to align or hold the needle in a vertical plane aligned with the axis.

The forward end 58 of the needle block may be angled forward from the needle (as opposed to vertically or backwards as in the embodiment shown) so that application of a forward directed force or motion on the needle block will urge the arm 36 toward the needle 16 and not away from the needle.

The distance 50 between the rearmost end 52 of arm 36 and the rear wall is preferably only slightly greater than the length 48 of the needle block. This aids in holding the needle block snuggly in the retracted position and also substantially eliminates a gap between the front end 58 of the needle block and the free end of the arm 36.

It will be noted that the needle guard includes side walls 62 that extend alongside the arm 36 and extend above the arm, so limiting access to the arm.

However, the walls 62 are sufficiently short such that a user can readily engage the top 64 of the needle block. Thus a user can withdraw the needle block 18 into the needle guard and then push the top 64 of the needle block toward the forward end 20 to satisfy themselves that the needle block is locked in position.

The needle block 19 is generally rectangular in cross section, as seen in FIGS. 9 to 11 with curved side walls 20 to aid a user in gripping the needle block 18. The exact shape of the needle block is not critical and needle block may have a generally circular shape in end view.

The needle guard preferably has an aperture forming assembly 70 comprising wall 30 and arms 32. The aperture forming assembly 70 is preferably formed integrally with the hollow portion 26. Preferably, the aperture forming assembly 70 is attached to hollow portion 26 via hinge 66 with snap arms 68 engaging in apertures 72 in hollow portion 26 to secure the assembly in position against accidental removal.

Whilst side wall 62 are formed with a gap 72 between their lower edge and the base wall 38, this is not essential and side walls 62 may be contiguous with base wall 38

In the embodiments of FIGS. 1 to 14, the bottom 38 is shaped and the needle block pivots at its front end against the bottom 38 under the force applied by the arm 36.

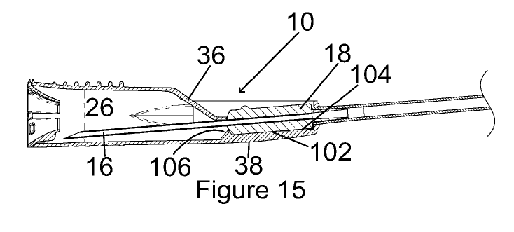

FIG. 15 shows a needle guard 100 according to another embodiment. This embodiment is substantially the same as the first embodiment except as described below and the same numerals are used for like parts.

In the needle guard 100 the bottom 38 in the retaining portion is shaped to present a sloping upper surface 102. When the needle block 18 is fully withdrawn into the retaining portion its lower surface 104 aligns with the surface 102 and aligns the needle block and needle 16 to the angled position shown. The surface sloping 102 thus acts as a deflector or deflector surface of the needle assembly 12. The arm 36 may remain deflected when the needle block is in this position and so apply a downwards force to the needle. Alternatively, the arm 36 may lie against or near to the needle without needing to apply a deflecting force to the needle. Upwards movement of the needle will be resisted by the arm 36 deflecting. Forwards motion will be prevented by the arm 36 lying in front of the needle block.

The bottom 38 may have a raised lip 106 at the front of the angled surface 102 to further limit forward motion of the needle block once in the retaining portion. The lip 106 may be sized so that as the needle block is withdrawn it engages the top of the lip 106 and is retained generally parallel to the needle guard axis as the needle is retracted through the aperture 24. As an alternative the bottom 38 may be provided with a recess and the needle block with a complementary protrusion (or vice versa) that, in the retracted position, engages in the recess and acts to retain the needle block in the retracted position.

Whilst the preferred embodiment has a flexible arm that engages the needle, it will be appreciated that the invention may be implemented by a flexible arm that engages the needle block, such as an upper surface, to cause or maintain a downward rotation of the needle block. The needle block may be provided with a protrusion or recess that the arm engages with to prevent forward movement from the retracted position.

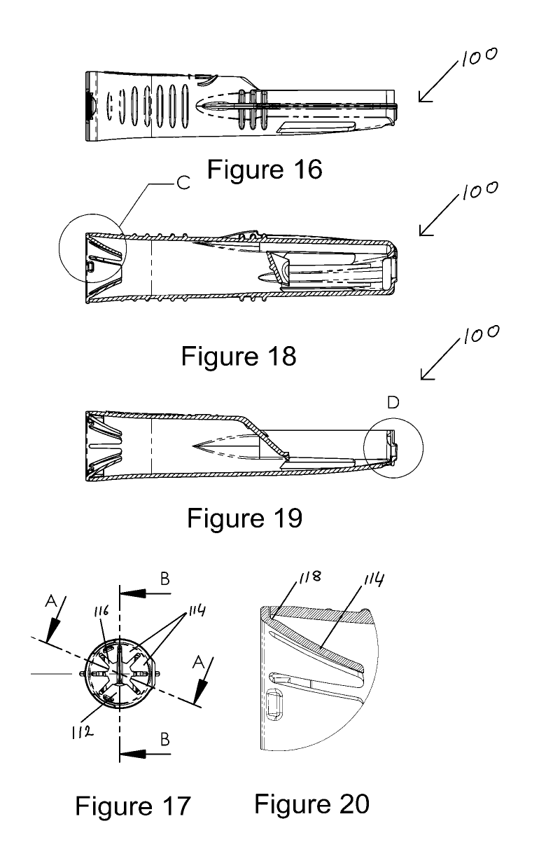

FIGS. 16 to 20 show a needle guard 100 having an alternate aperture arrangement. The arrangement of the locking/deflector arm is substantially as the other embodiments. In this embodiment the aperture 110 is formed of a relatively rigid wall 112 and six, more flexible, arms 114. Excluding allowance for the gaps or slots 116 between adjacent arms 114 and wall 112, the wall 112 extends around the aperture 110 about 90 degrees and the arms, in total, extend over about 270 degrees. In this embodiment the six arms 114 are substantially identical but need not be. To aid flexing of the arms 114, a hinge or line of weakness 118 is provided.

The aperture arrangement of the FIGS. 26 to 20 embodiment may be used with the other embodiments.

Unless the context clearly requires otherwise, throughout the description and any claims the words “comprise”, “comprising”, and the like are to be construed in an inclusive sense as opposed to an exclusive or exhaustive sense; that is to say, in the sense of “including, but not limited to”.

The features of the invention described or mentioned in this document may be combined in any combination of features where features are not mutually exclusive.

It will be apparent to those skilled in the art that many obvious modifications and variations may be made to the embodiments described herein without departing from the spirit or scope of the invention.

1. A needle guard for use with a needle assembly having a needle extending from a needle block having a forward end, the guard comprising:
a hollow sleeve portion having a front end;
wherein the hollow sleeve portion is relatively rigid;
an aperture at the front end through which the needle assembly may be retracted to a retracted position, in which the free end of the needle lies within the hollow sleeve portion;
wherein the aperture is at least partially defined by at least one first wall and at least first one arm;
wherein the at least one first wall is relatively rigid and/or immovable and the at least one first arm is relatively flexible and/or movable;
wherein the aperture is smaller than the needle block and wherein the at least one first arm may be flexed and/or moved to increase the size of the aperture to allow the needle and needle block pass through the aperture;
a retaining portion into which at least a part of the needle assembly may be withdrawn to a retracted position;
at least one locking mechanism to retain the needle assembly in the retaining portion;
at least one deflector that cooperates the needle assembly to deflect the needle away from the aperture when in the retracted position,
wherein in the retracted position the end of the needle lies behind the at least one first wall.

2. (canceled)

3. A needle guard as claimed in claim 28 wherein the at least one deflector comprises includes a surface portion of the retaining portion complementary to the surface of the needle block so that when the needle block bears against the surface portion the free end of the needle is located away from the aperture and behind the at least one first wall.

4. (canceled)

5. (canceled)

6. (canceled)

7. A needle guard as claimed in claim 3 including at least one flexible retaining arm that, when the needle assembly is in the retracted position, engages with, bears against or lies adjacent to a portion of the needle and/or the needle block to resist movement of the needle block away from the deflector surface.

8. A needle guard as claimed in claim 7 wherein, when the needle assembly is in the retracted position, the at least one flexible retaining arm applies a deflecting force to the needle assembly.

9. A needle guard as claimed in claim 7 wherein the at least one flexible retaining arm comprises at least part of the at least one locking mechanism, the at least one flexible retaining arm includes at least one locking portion that lies forward of a part of the needle block and limits forward movement of the needle block.

10. (canceled)

11. A needle guard as claimed in claim 9 wherein the at least one locking portion includes an end of at least one flexible arm.

12. A needle guard as claimed in claim 9 wherein the at least one flexible retaining arm has a needle engagement surface that engages with, bears against or lies adjacent to the needle.

13. A needle guard as claimed in claim 12 wherein the needle engagement surface is curved.

14. (canceled)

15. (canceled)

16. A needle guard as claimed in claim 9 including a retaining member to assist retention of the needle assembly in the retaining portion.

17. A needle guard as claimed claim 16 wherein the retaining member comprises a protrusion that is located forward of the forward end of the surface portion and extends above the surface portion needle block.

18. A needle guard as claimed in claim 26 wherein the at least one first wall comprises a single wall.

19. A needle guard as claimed in claim 18 wherein in the at least one first arm comprises at least three arms.

20. A needle guard as claimed in claim 19 wherein each arm extends at least about 45 degrees around the aperture.

21. A needle guard as claimed in claim 20 wherein the at least one first wall extends at least about 90 degrees around the aperture.

22. A needle guard as claimed in claim 21 wherein the at least one first wall extends about 180 degrees around the aperture.

23. A needle guard as claimed in claim 1 wherein the at least one first wall extends rearwards and inwards.

24. (canceled)

25. (canceled)

26. A needle guard as claimed in claim 23 wherein the at least one first arm extends rearwards and inwards.

27. A needle guard as claimed in claim 22 wherein the at least one first arm extends about 180 degrees around the aperture.

28. A needle guard as claimed in claim 21 wherein the at least one arm extends about 270 degrees around the aperture.
